(19) United States
(12) Patent Application Publication
(10) Pub. No.: US 2008/0149115 A1
(43) Pub. Date: Jun. 26, 2008

(54) SURGICAL STATION FOR ORTHOPEDIC RECONSTRUCTION SURGERY

(75) Inventors: Brian Hauck, Windsor, CA (US); Heber Saravia, Santa Rosa, CA (US); Christopher Staudenmayer, Santa Rosa, CA (US); Kai U. Mazur, Santa Rosa, CA (US)

Correspondence Address: SHAY GLENN LLP, 2755 CAMPUS DRIVE, SUITE 210, SAN MATEO, CA, 94403, US

(73) Assignee: SONOMA ORTHOPEDIC PRODUCTS, INC., SANTA ROSA, CA (US)

(21) Appl. No.: 11/944,385

(22) Filed: Nov. 21, 2007

Related U.S. Application Data
(60) Provisional application No. 60/866,920, filed on Nov. 22, 2006.

(51) Int. Cl. A61F 5/37; A61F 5/00

(52) U.S. Cl. 128/878; 602/33

(57) ABSTRACT

A limb supporting apparatus adaptable for use during orthopedic surgery on a patient's limb having a platform base; an appendage rest supported by the platform base and adapted to immobilize the limb; an access port supported by the platform base; and a curved cannula supported by the access port and adapted to facilitate access to target bone during orthopedic surgery. Another aspect of the invention provides a method of performing an orthopedic procedure on a limb including the steps of immobilizing the limb on an appendage rest of a platform base; aligning a curved cannula supported by an access port with a target bone of the limb; and performing an orthopedic procedure on the target bone through the cannula.

### CROSS-REFERENCE

This application claims the benefit of U.S. Provisional Patent Application No. 60/866,920 entitled “Surgical Station for Orthopedic Reconstructive Surgery,” filed Nov. 22, 2006, which disclosure is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The invention relates to a limb holding device designed to immobilize, retract, and stabilize the upper extremities of the human anatomy during reconstructive orthopedic surgery. An arc cannula can be provided as a guide for using burrs and drills to access target tissue or bone.

Currently available limb supporting devices for arthroscopic surgery include that described in U.S. Pat. No. 5,027,799 to Laico et al. The device is a limb holding device designed to be attached to an operating table to immobilize a patient's arm or leg during arthroscopic surgery or the like. U.S. Pat. No. 5,785,057 to Fischer for Medical Positioning Devices describes an apparatus for positioning an upper appendage portion of a body of a patient with respect to an operating room table having a mattress covering at least a portion of the operating room table and a leaf portion moveable out of a coplanar position with respect to a remaining portion of the operating room table during a surgical procedure. U.S. Pat. No. 6,221,036 to Lucas for Support for a Limb of a Body describes a support having a sheet of flexible material with longitudinal looks running along the sides of the sheet. The loops are releasably and securely attached with rods to the sides of a base for the support.

### SUMMARY OF THE INVENTION

The invention is directed to an immobilization device, or surgical station, that is non-invasive, non-implantable and skin contacting only. The device immobilizes, retracts and stabilizes the limbs of the human anatomy during orthopedic surgery. In addition, tension applied to the extremities is used to help reduce and stabilize fractures during surgery. An arc cannula acts as a guide to the physician or surgical technician when using tools to access target tissue or bone or implanting devices within the bone, such as those tools and devices described in the following copending applications: U.S. application Ser. No. 60/866,976 filed Nov. 22, 2006, to Phillip Jobson for SURGICAL TOOLS FOR USE IN DEPLOYING BONE REPAIR DEVICES; U.S. application Ser. No. 60/867,011 filed Nov. 22, 2006 to Phillip Jobson for BONE REPAIR IMPLANT WITH CENTRAL RATCHETING GUIDEWIRE; PCT Appln. No. PCT/US06/18704 for Minimally Invasive Actuable Bone Fixation Devices, Systems and Methods of Use by Charles L. Nelson et al.; U.S. patent application Ser. No. 11/383,279 filed May 15, 2006 by Charles Nelson et al. for Methods of Using Minimally Invasive Actuable Bone Fixation Devices; U.S. patent application Ser. No. 11/383,275 filed May 15, 2006 by Charles Nelson et al. for Minimally Invasive Actuable Bone Fixation Devices Having a Retractable Interdigitation Process; and U.S. patent application Ser. No. 11/383,269 filed May 15, 2006 by Charles Nelson et al. for Minimally Invasive Actuable Bone Fixation Devices.

In some embodiments, the invention is a surgical station that allows immobilization, retraction, preparation, and articulation of a target anatomy during orthopedic surgery. The station provides straps and tie down anchors that rigidly immobilize the anatomy. It has a platform that slides in the x-y plane, along positive and negative x and positive and negative y axes. The station provides rotation of the limb about the x axis.

One aspect of the invention provides a limb supporting apparatus adaptable for use during orthopedic surgery on a patient's limb, comprising a platform base, an appendage rest supported by the platform base, an access port, and a curved cannula supported by the access port. The platform base has a plurality of connection elements adapted to connect components to the platform base at a plurality of connection locations. The appendage rest and access port and other accessories are adapted to connect to the platform base at a plurality of connection locations.

In some embodiments the appendage rest comprises an appendage rest base and an appendage rest platform, wherein the appendage rest platform engages the appendage rest base, such that the platform may rotate. The appendage rest base has a plurality of connection elements adapted to connect the appendage rest to a plurality of connection locations on the platform base. The appendage rest platform may have radiopaque markers provided to assist a surgeon in guiding tools and devices within the bone.

In some embodiments a longitudinal stabilizer engages a plurality of locations along the length of the platform base distal to the appendage rest. The longitudinal stabilizer provides traction to an end of the patient's limb.

In some embodiments, the access port comprises an access port base that engages the platform base, a height adjustable cannula access carrier, and a cannula access carrier that rotates coplanar to the platform base. The access port base engages the platform base at a plurality of engagement locations.

Another aspect of the invention provides a method of performing an orthopedic procedure as follows: immobilize the limb on an appendage rest of a platform base; align a curved cannula supported by an access port with a target bone; and perform a procedure on the target bone through the cannula. A distal end of the limb may be aligned with a distal end of the appendage rest. The appendage rest connection elements are engaged to fewer then all of the connection elements adapted to the platform base. The limb is secured to the immobilizing support apparatus in a plurality of locations along the length of the limb, and traction may be applied to the limb. The appendage rest platform may be rotated. The access port engages the platform base at a plurality of engagement locations. In some embodiments, alignment of the cannula comprises adjusting the height of a cannula access carrier to vertically align the cannula and rotating the access carrier in the horizontal plane to align the cannula with the target. The invention allows surgeons to plan, adjust and visualize the trajectory of the access port in the bone before it is cut while at the same time provides means for immobilization of the patient's limb.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustration of a surgical station in use.

FIGS. 2a-2d are: a bottom view of a surgical station access port; a top view of a surgical station access port; an overall view of a surgical station access port; a section of a surgical station access port.

FIG. 3a is an appendage rest for a surgical station

FIG. 3b is a section of an appendage rest of a surgical station.

FIG. 4 is an overall view of a longitudinal stabilizer portion of the surgical station.

FIG. 5 is an overall view of an assembled surgical station having all of the components featured in FIGS. 1-4.

FIG. 6 is an image of a portion of an arm on a surgical station of the invention with radiopaque markers used for guidance visible to a user.

FIG. 7 is a view of a portion of an arm on a surgical station of the invention with a cannula engaging the target.

### DETAILED DESCRIPTION OF THE INVENTION

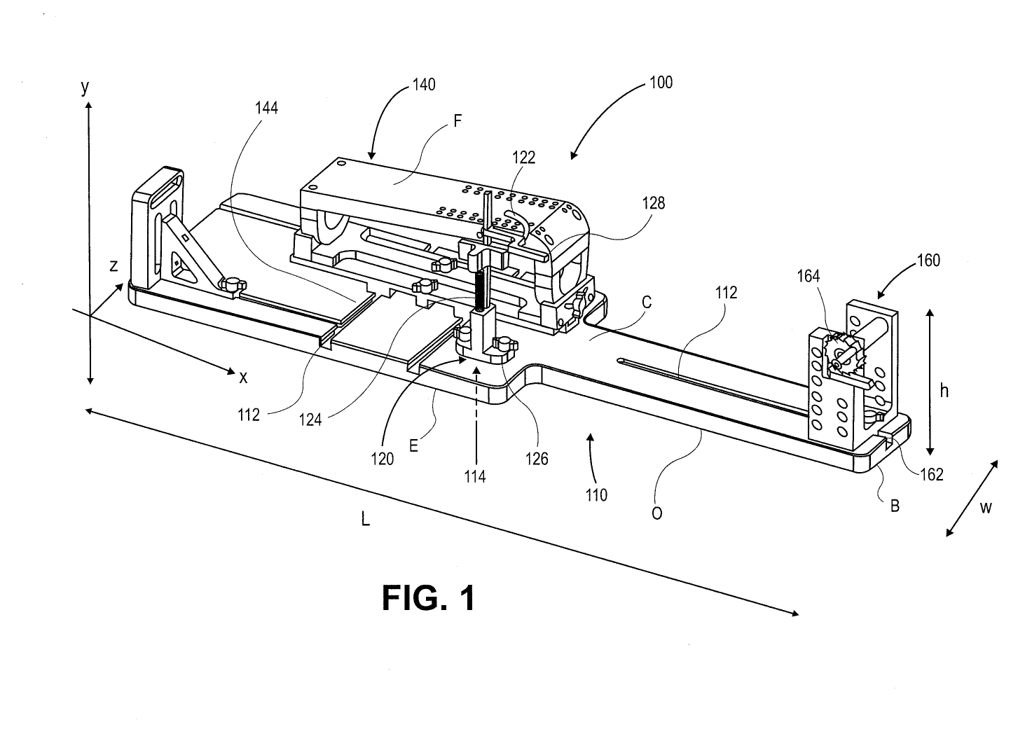

FIG. 1 illustrates a surgical station 100 for use in orthopedic reconstructive surgery. The surgical station 100 has a platform 110 or platen. The station is positioned within a plane, shown by the x-y-z coordinates, and is substantially rectangular in length 1 and width w. Additionally, the station 100 is adapted to provide a plurality of components each of which can achieve its own optimal height h. The station also has a side that is proximal A to the patient (i.e., relatively closest to the core of a patient's body) and distal B to the patient (i.e., a side that is furthest away from the core of the patient and toward which the end of the patient's engaged appendage approaches). Additionally, there is an interior side D and an exterior side C. These orientations are provided as a guide to appreciating the location of a particular component of the device relative to, for example, the patient, the operating table, and/or the surgeon.

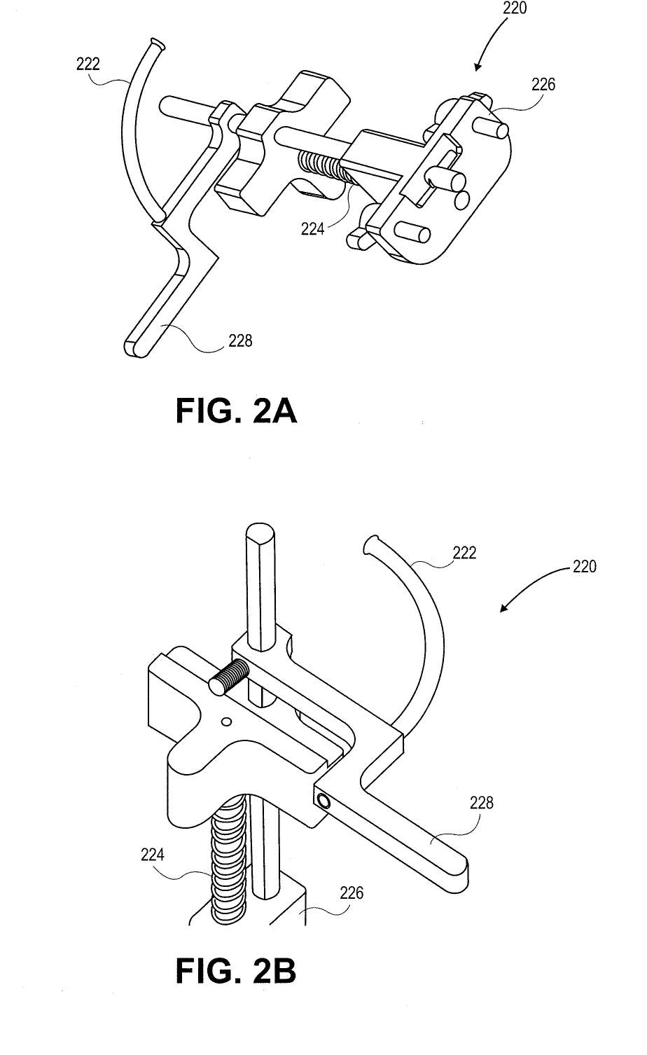

As depicted in FIG. 1, an example of a surgical station according to the invention, the platform is adapted to such that its lower side E rests on a surface, such as an operating table. A plurality of channels 112, 112′ and apertures 114 are provided to facilitate connecting adjustable and relocatable pieces onto the platform. An access port 120, shown in more detail in FIG. 2, is provided that is adapted to engage the platform 10. The access port 120 is configured to be adjustable in height by, and to provide cannula access 122/222 which is adapted to engage a target portion of the anatomy of a patient to facilitate access to target bone during orthopedic reconstructive surgery. A threaded shaft 124 is provided that engages an access port base 126 and a cannula access carrier 128. The shaft engages a threaded aperture in each of the base 126 and carrier 128 to facilitate turning the shaft 124.

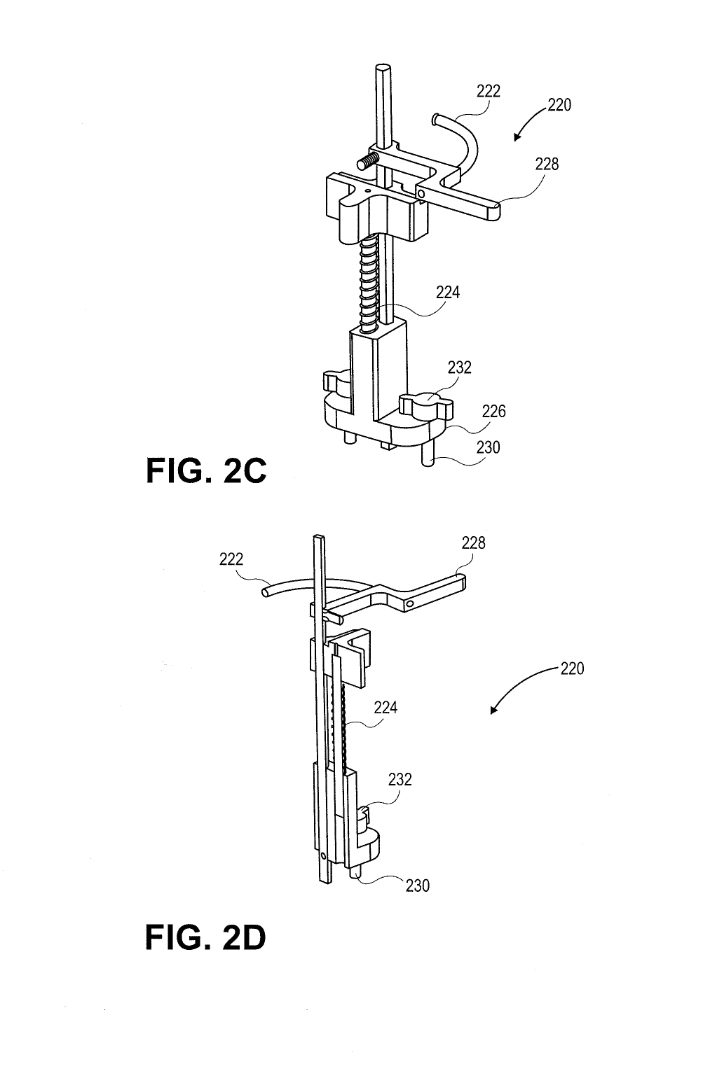

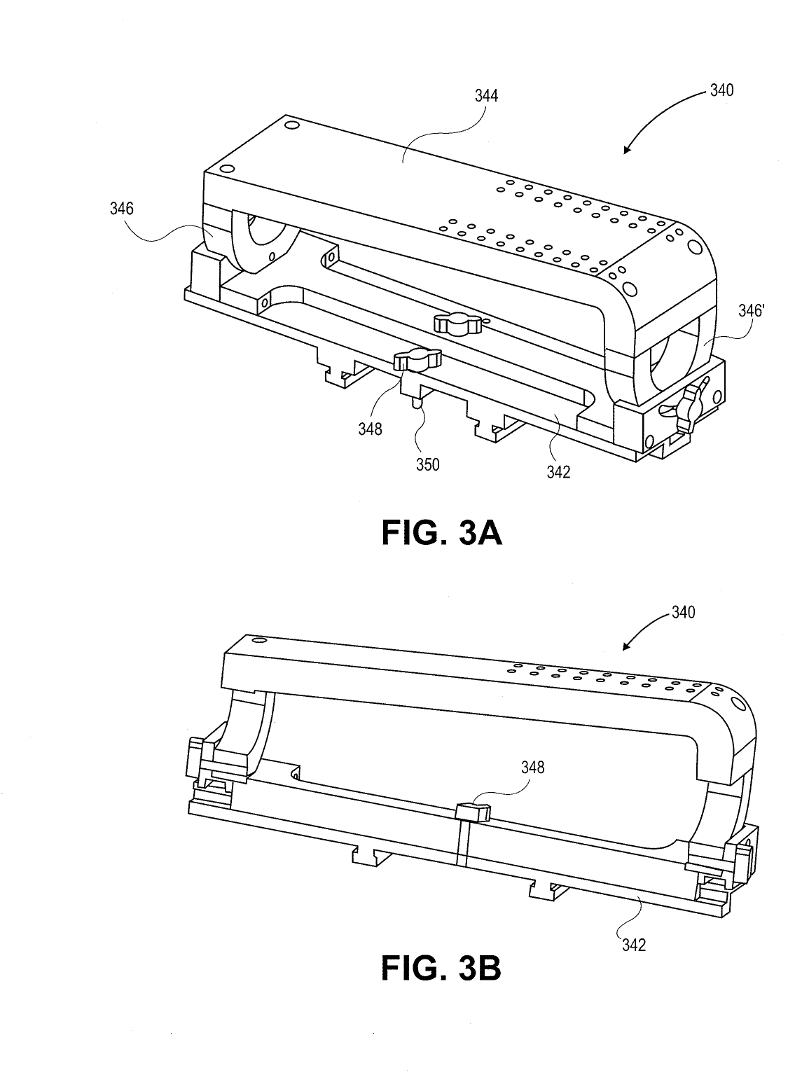

An appendage rest 140 is also provided, shown in more detail in FIG. 3, which supports, for example, the arm of a patient undergoing surgery. The rest 140 has a base 342 adapted to engage the platform, such as by using a tongue-and-groove arrangement. As depicted the rest 140 slides onto the surgical station platform with the tongue-and-groove engaging mechanism at a non-parallel angle to the limb of the patient being supported and/or the long axis of the anatomical rest.

A longitudinal stabilizer 160, such as a finger traction device, is provided on the platform 110 at or near an end. The stabilizer engages the platform using a suitable mechanism, such as a tongue-and-groove engaging mechanism (162, 112) that is adapted to be slidably positioned along the length of the platform 110. The stabilizer is adapted to secure an end of a limb of a patient while maintaining sufficient tension to avoid rotational movement. A ratchet mechanism 164 is an example of one mechanism provided to engage the patient and provide sufficient tension.

Turning now to FIGS. 2a-2d, the surgical station access port 220 is depicted. A curved cannula 222 is provided that is adapted to extend from the access port 220 toward the patient to deploy and support tools and surgical devices. As described above, the access port 220 is adjustable in height in order to optimally position the curved cannula during use. The height is adjusted with the threaded shaft 124. Engaging pins 230 are provided that engage the platform 110. Once the base 226 is in place and the pins 230 are positioned within apertures on the base, the access port 220 can be locked in place by engaging one or more knobs 232. In an alternative embodiment, the pin 230 can be a threaded screw that engages a threaded female aperture on the base. Other configurations and adjustment mechanisms can be employed without departing from the scope of the invention.

FIG. 3 illustrates an appendage rest 340. The rest 340 has an appendage rest base 342 adapted to engage the platform 110, such as by using a tongue-and-groove mechanism. An immobilization and stabilization appendage rest platform 344 is adapted to engage the base. Radiopaque markers can be provided, e.g. embedded within the platform, to facilitate determination of target pathway within the immobilized bone for tools and or devices deployed. The platform 344 can further have curved supports 346, 346′ that engage the base 342 and allow the platform to be rotated such that the horizontal plane encompassing the appendage engaging surface of the platform may or may not be parallel to the horizontal plane encompassing the base 342. Threaded knobs 348 have tips 350 that are pressed against platform 110 to lock the platform in place.

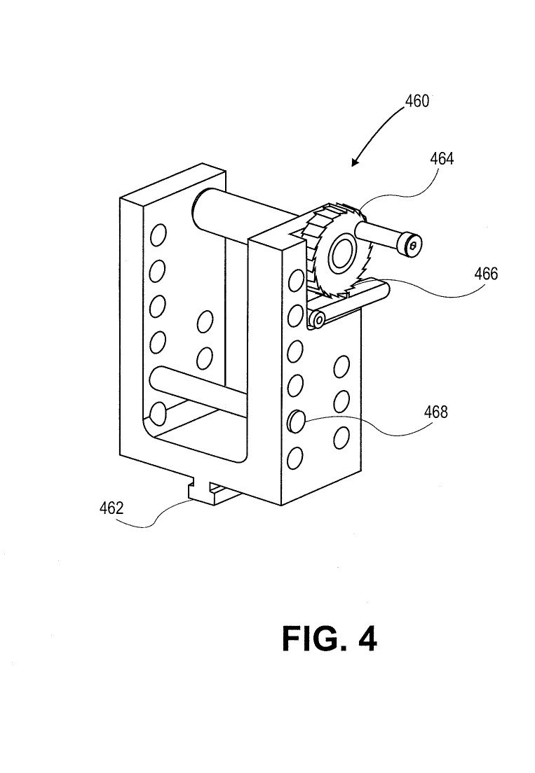

As illustrated in FIG. 4 a longitudinal stabilizer 460 is provided. The stabilizer engages the platform at or near an end with a suitable mechanism, such as a T-slot feature (the transverse 462 portion depicted). A ratchet mechanism 464 is provided that engages a pawl 466 to enable the system to achieve linear motion of the engaged appendage in a direction to stabilize it. A post 468 is provided that is configured to be positioned to engage one set of a plurality of matched apertures to enable the height of the post to be adjusted for a particular patient.

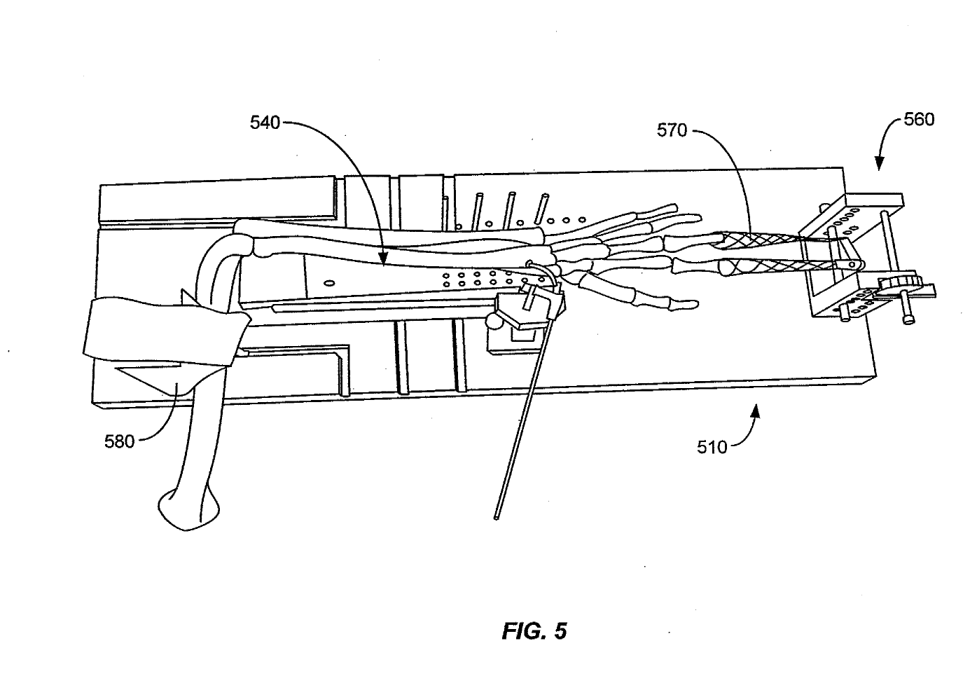

FIG. 5 illustrates a surgical station with an appendage positioned on an appendage rest 540 therein wherein the fingers are engaged with finger traps 570 that are connected to the longitudinal stabilizer 560 and secured using the ratchet mechanism to pull the fingers toward the stabilizer. (The tissue surrounding the bones has been omitted from FIG. 5 for purposes of illustration.) An additional appendage anchor 580 is provided which assists in securing the appendage at a proximal location to the surgical station. Thus the appendage may be secured at a relatively distal location by securing the distal part of the limb to longitudinal stabilizer and at a proximal most section (depicted as the upper arm of a skeleton in FIG. 5) to the proximal securement device, which is also adapted to adjustably engage the platform 510.

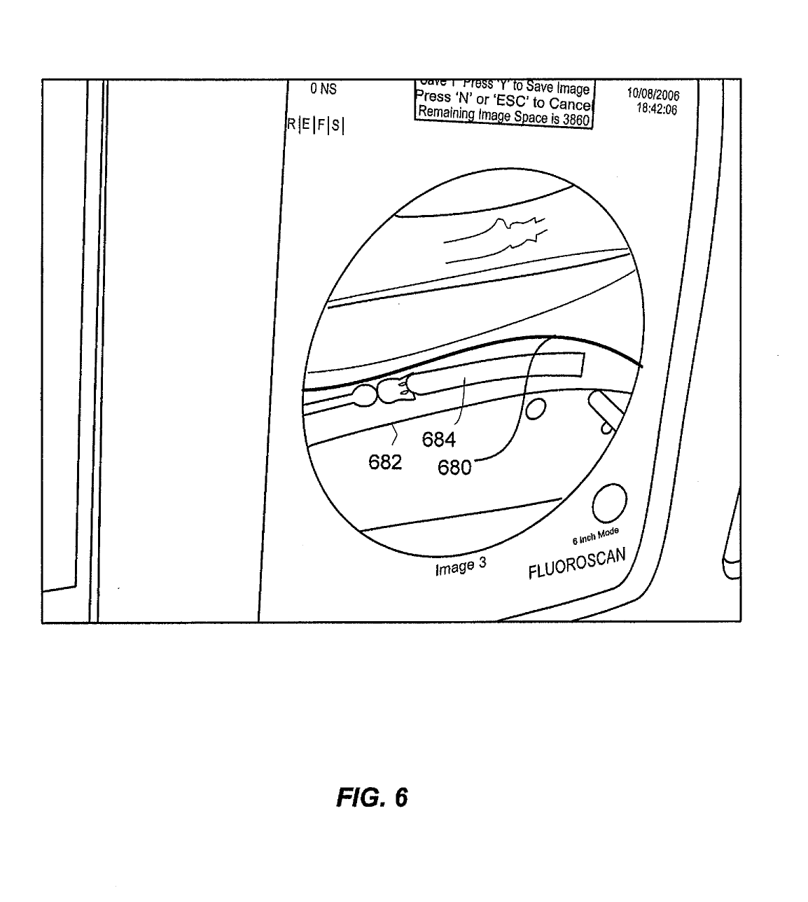

FIG. 6 is an image of a portion of an arm positioned on a surgical station for orthopedic surgery. A radiopaque marker 680 used to assist a surgeon in guiding tools and devices (such as implant 684) within the bone 682 are depicted. The curve of the marker enables the surgeon to match the shape of a metaphyseal hub component of a repair device 684 (such as that described in U.S. application Ser. No. 60/867,011), during the surgical procedure and to predict the trajectory the reamer will follow as guided by access port 220. From the above descriptions it can be appreciated that the surgery station permits adjusting the height and angle of various components relative to the base of the station. Additionally, translation of the patient's appendage can be achieved by, for example, adjusting the position (i.e., the plane in which it sits) of the appendage rest platform relative to the position of the base.

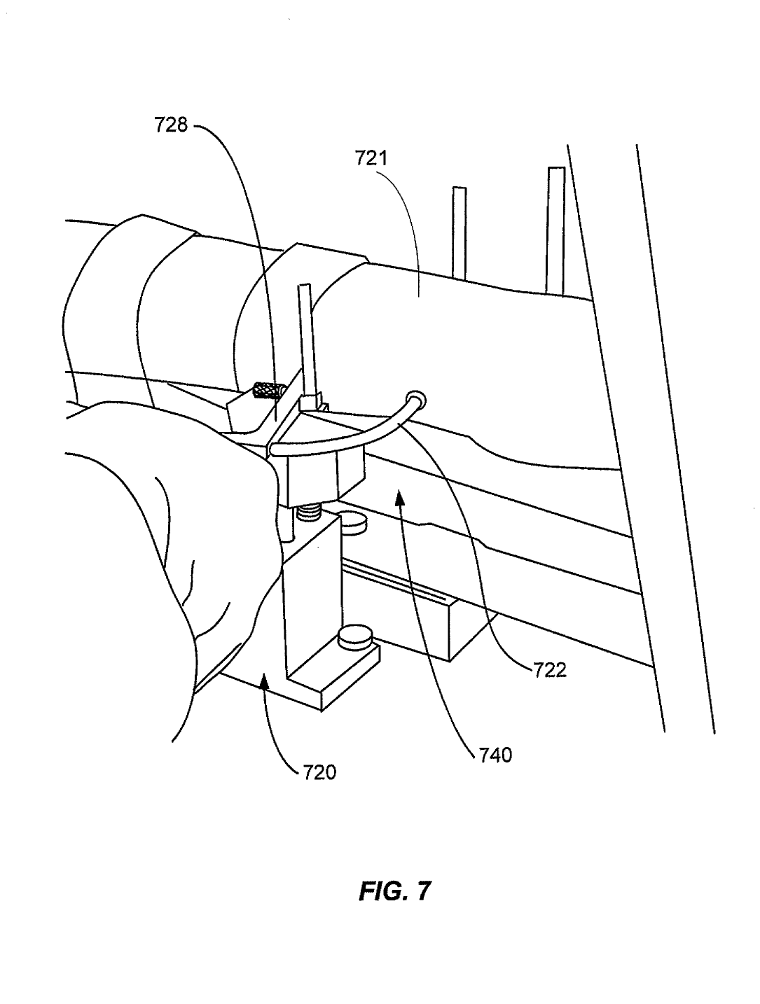

FIG. 7 illustrates an access port 720 positioned on a surgical station in relation to an arm 721 that is immobilized on and secured to an appendage rest 740 wherein a cannula access carrier 728 engages a cannula 722 with a target portion of the arm. The cannula is used to guide reaming and drilling tools over a predetermined trajectory and it can also be used to pass tools and devices to and into the arm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A limb supporting apparatus adaptable for use during orthopedic surgery on a patient's limb comprising:
a platform base;
an appendage rest supported by the platform base and adapted to immobilize the limb;
an access port supported by the platform base; and
a curved cannula supported by the access port and adapted to facilitate access to target bone during orthopedic surgery.

2. The limb supporting apparatus of claim 1, wherein the platform base comprises a plurality of connection elements adapted to connect components to the platform base at a plurality of connection locations.

3. The limb supporting apparatus of claim 1, wherein the appendage rest comprises a plurality of connection elements adapted to connect the appendage rest to a plurality of connection locations on the platform base.

4. The limb supporting apparatus of claim 1, wherein the appendage rest comprises an appendage rest base and an appendage rest platform.

5. The appendage rest of claim 4, wherein the appendage rest platform engages the appendage platform base, such that the plane of the platform may rotate out of parallel to the horizontal plane encompassing the platform base.

6. The limb supporting apparatus of claim 1, wherein the appendage rest comprises a plurality of connection elements adapted to connect the appendage rest to a plurality of connection locations on the platform base.

7. The limb supporting apparatus of claim 1, wherein a longitudinal stabilizer is provided distal to the appendage rest on the base, the stabilizer being adapted to apply traction to an end of the patient's limb.

8. The limb supporting apparatus in claim 6 wherein the longitudinal stabilizer is adapted to engage the platform to a plurality of locations along the length of the platform, distal to the appendage rest.

9. The limb supporting apparatus of claim 1 wherein the access port comprises:
an access port base that engages the platform base; and
a height adjustable cannula access carrier; and
a cannula access carrier that rotates coplanar to the platform base.

10. The access port base of claim 9 wherein the platform base comprises a plurality of access port base engagement locations.

11. The limb supporting apparatus of claim 1 wherein the appendage rest platform comprises a radiopaque marker to assist a surgeon in guiding tools and devices within the target bone.

12. A method of performing an orthopedic procedure on a limb comprising:
immobilizing the limb on an appendage rest of a platform base;
aligning a curved cannula supported by an access port with a target bone of the limb; and
performing an orthopedic procedure on the target bone through the cannula.

13. The method of claim 12 further comprising aligning a distal end of the limb with a distal end of the appendage rest.

14. The method of claim 12 further comprising engaging a plurality of connection elements on the appendage rest to fewer then all of the connection elements adapted to the platform base.

15. The method of claim 12 further comprising securing the limb to the immobilizing support apparatus in a plurality of locations along the length of limb.

16. The method of claim 12 further comprising rotating the appendage rest.

17. The method of claim 12 further comprising applying traction to the patient's limb.

18. The method of claim 12 further comprising engaging the access port with the platform base at a plurality of engagement locations.

19. The method of claim 12 wherein the aligning step comprises adjusting the height of a cannula access carrier with respect to the access port to vertically align the cannula with the target bone.

20. The method of claim 12 wherein the aligning step comprises rotating the cannula access carrier in the horizontal plane to align the cannula with the target bone.
